(19) United States
(12) Patent Application Publication
(10) Pub. No.: US 2007/0093911 A1
(43) Pub. Date: Apr. 26, 2007

(54) Soft tissue implant such as breast implant, calf muscle implant or the like

(76) Inventors: Helmut Fricke, Meinersen (DE); Hanngorg Zimmermann, Roßtal (DE)

Correspondence Address: MCGLEW & TUTTLE, PC, P.O. BOX 9227, SCARBOROUGH STATION, SCARBOROUGH, NY, 10510-9227, US

(21) Appl. No.: 10/554,792

(22) PCT Filed: Apr. 20, 2004
(86) PCT No.: PCT/EP04/04170

§ 371 Date: Oct. 27, 2005

(30) Foreign Application Priority Data
Apr. 28, 2003 (DE) 20306637.5

(51) Int. Cl. A61F 2/02; A61F 2/08; A61F 2/12

(52) U.S. Cl. 623/23.72; 623/8; 623/14.13

(57) ABSTRACT

A soft tissue implant such as a breast implant, calf muscle prosthesis and the like, comprises an external shell-type envelope (1) of flexible plastic material, in particular of silicone; a liquid to viscous filler material (5) contained in the envelope (1); and a metal-containing, biocompatible, continuous coating (9) on the outside (7) of the envelope (1).

The invention relates to a soft tissue implant comprising an external shell-type envelope of flexible plastic material, in particular silicone, and a fluid to viscous filler material contained in the envelope.

Soft tissue implants of the generic type are used for example in the form of breast implants in the field of plastic surgery after breast amputations or in cosmetic surgery for breast augmentation. Further fields of application of these soft tissue implants are calf muscle prostheses or cheek, nose, gluteal muscle, testicular or brachial muscle implants.

Problems with these soft tissue implants are posed by the fact that the human body will try, by a reaction of encapsulation, to separate the implant, perceiving the plastic material on the surface as foreign substance. To this end, the human body deposits comparatively hard, rigid tissue by which to encapsulate the implant at least partially or locally. Even inflammatory reactions to the inserted soft tissue implant occur from time to time.

It is an object of the invention to embody a soft tissue implant of the species in such a way that any significant reactions of rejection by the human body do not occur, which reliably precludes the described encapsulation of the implant.

This object is attained by the features specified in the characterizing part of claim 1, according to which a metal-containing, biocompatible, continuous coating is applied to the outside of the implant envelope.

Preferably, this coating is a titanium-containing coating of a thickness of less than 2 μm, preferably of 5 to 700 nm. This coating is flexible, completely participating in the motions of the envelope without any signs of detachment. Furthermore, this coating, which is applied to the plastic material that constitutes the envelope, excels by adhesive and frictional strength.

The metal-containing coating preferably consists of a compound of the formula TiₐO_{b}C_{c}, with
a=0.025 to 0.9,
b=0.025 to 0.7 and
c=0.2 to 0.9
   applying. Optionally, the titanium constituents can be replaced by tantalum, niobium, silver, zirconium and hafnium. Nitrogen and boron may be further elements in the compound.

In addition to the metal-containing coating on the outside of the envelope, a metal-containing, continuous coating can additionally be applied to the inside of the envelope. The coating has been found not only to ensure biocompatibility of otherwise incompatible plastics, but simultaneously to function as a diffusion barrier to various ions and molecules. In this regard, the coating on the inside and outside of the envelope of plastic material has the effect that the plasticizers, which are regularly contained in the plastic material, cannot gradually escape from the material. Consequently, the flexibility of the plastic material is maintained even for a prolonged period of time. On the other hand, the coating prevents the filler material from escaping from the envelope into the human body; the filler material may be viscous silicone gel, physiological sodium chloride solution or water. In this regard, the in particular bilateral, metal-containing coating strongly improves the impermeableness of the soft tissue implant so that again silicone gel can be used more and more often as filler material. Upon leakage of the envelope and escape into the human body, silicone gel causes serious health problems, which is not the case with water or physiological sodium chloride solution. However, applying silicone gel is preferred, its consistency bearing greater similarity to body tissue. Moreover, with the viscosity of silicone gel exceeding that of water or sodium chloride solution, any kinking damages are avoided in the vicinity of narrow curves of the envelope, for example where the outwardly bulged area of a breast implant passes into the distal base area.

Finally, both coatings can be smooth or rough so that the surface quality of the implant can be set regardless of the envelope.

Details of the invention will become apparent from the ensuing description of an exemplary embodiment of the invention, taken in conjunction with the drawing, in which

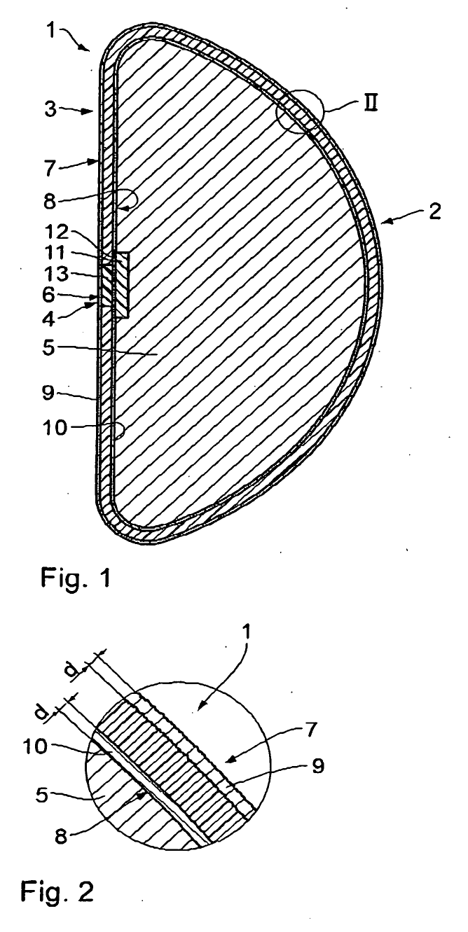

FIG. 1 is a diagrammatic, radial, sectional view of a breast implant; and

FIG. 2 is a diagrammatic sectional view, on an enlarged scale, of the detail II according to FIG. 1.

As seen in FIG. 1, the illustrated breast implant comprises an external shell-type envelope 1 which conforms to the natural shape of the breast and is made of flexible silicone material. The envelope 1 is comprised of an area 2 which bulges in the proximal direction and a substantially flat, rounded base area 3. This base area 3 is centrally provided with a hole 4 which, once the filler material 5 has been inserted in the envelope 1, is tightly closed by a two-piece seal 6. By way of an adhesive ring 12, an inward seal 11 is glued on to the inside 8 of the envelope 1 around the hole 4. The remaining external impression is then closed by an external seal insert 13. The filler material 5 is a viscous silicone gel, entirely filling the interior of the envelope 1 in such a way that it is not inflated to tautness and does not sag or collapse.

As seen in FIG. 2, the outside and inside 7, 8 of the envelope 1 possess a continuous, biocompatible coating 9, 10 of a titanium-containing material of the formula TiₐO_{b}C_{c}. The constituent portions a, b and c correspond to the ranges mentioned in the introductory part. A coating of that type has proved to be absolutely biocompatible. Owing to the continuity of the coating, the plastic material of the envelope 1 is no longer perceived as such by the human body. The biocompatibility is therefore comparable to implants which are completely manufactured from a titanium alloy and which are widely accepted in medical engineering.

Preferred thicknesses d of the coatings 9, 10 are in a range of 5 to 700 nm, with coating thicknesses of approximately 50 nm having proved to possess special adhesive and frictional strength on the one hand and to be sufficiently flexible and ductile on the other, the coating thus being able without any damages to participate in any stretching of, and strain on, the envelope.

Titanium-containing coatings of that type and the technique of applying them to flexible plastic substrates are fundamentally known from the prior art, for example from EP 0 897 997 B1.

As outlined in FIG. 2, the outside 7 of the envelope 1 is slightly rough only on the surface of the substrate. This textured surface provides for the risk of capsular fibrosis to be reduced to a minimum.

1. A soft tissue implant, such as a breast implant, calf-muscle prosthesis and the like, comprising;
an external, shell-type envelope (1) of flexible plastic material, in particular silicone;
a liquid to viscous filler material (5) contained in the envelope (1); and
a metal-containing, biocompatible, continuous coating (9) on the outside (7) of the envelope (1);
wherein a metal-containing continuous coating (10) is additionally applied to the inside (8) of the envelope (1).

2. A soft tissue implant according to claim 1, wherein the coating (9, 10) is a titanium-containing coating of a thickness of less than 2 μm, preferably of 5 to 700 nm.

3. A soft tissue implant according to claim 2, wherein the coating (9, 10) comprises a compound of the formula
TiₐO_{b}C_{c},
with a=0.025 to 0.9,
b=0.025 to 0.7 and
c=0.2 to 0.9
applying.

4. A soft tissue implant according to claim 1, wherein the outside (7) of the envelope (1) is smooth.

5. A soft tissue implant according to claim 1, wherein the outside (7) of the envelope (1) has a rough surface.

6. A soft tissue implant according to claim 1, wherein the filler material (5) is a viscous silicone gel.

7. A soft tissue implant according to claim 1, wherein the filler material (5) is a physiological sodium chloride solution or water.

8. (canceled)
